(19) United States
(12) Patent Application Publication
(10) Pub. No.: US 2013/0005999 A1
(43) Pub. Date: Jan. 3, 2013

(54) METHOD FOR REGENERATING TITANOSILICATE CATALYSTS

(75) Inventors: Tomonori Kawabata, Toyonaka-shi (JP); Masato Yoshioka, Yokosuka-shi (JP)

(73) Assignee: SUMITOMO CHEMICAL COMPANY, LIMITED, Tokyo (JP)

(21) Appl. No.: 13/635,345

(22) PCT Filed: Mar. 11, 2011
(86) PCT No.: PCT/JP2011/056491

§ 371 Date: Sep. 14, 2012

(30) Foreign Application Priority Data
Mar. 18, 2010 (JP) 2010-062106

(51) Int. Cl. C01B 39/00; C07D 301/12

(52) U.S. Cl. 549/523; 423/700

(57) ABSTRACT

A method for regenerating a titanosilicate catalyst, comprising a step of mixing a titanosilicate having reduced catalytic ability with a cyclic secondary amine, and a method for producing an oxirane compound, which comprises a step of reacting hydrogen peroxide with a C2-C12 compound having a carbon-carbon double bond, in the presence of a titanosilicate catalyst obtained by the regeneration method.

### TECHNICAL FIELD

The present invention relates to a method for regenerating titanosilicate catalysts.

### BACKGROUND ART

Titanosilicates have been known to be useful as catalysts for propylene oxide synthesis reaction from propylene, oxygen and hydrogen. When titanosilicates are continuously used as such catalysts, they reduce their catalyst activity or catalytic ability. Patent document 1 describes a method for regenerating titanosilicate catalysts with reduced catalytic ability, wherein a titanosilicate catalyst is contacted with acetonitrile or a mixture of water and acetonitrile at a temperature of 25° C. to 200° C.

### CITATION LIST

### Patent Literature

[Patent document 1] Japanese Unexamined Patent Application Publication No. 2009-233656

### SUMMARY OF INVENTION

### Technical Problem

It is an object of the invention to provide a regeneration method that can regenerate a titanosilicate having reduced catalytic ability.

### Solution to Problem

The following invention has been completed as a result of much diligent research on methods of regenerating titanosilicate catalysts, in light of the current situation. Specifically, the invention provides the following.

<1> A method for regenerating a titanosilicate catalyst, comprising a step of mixing a titanosilicate having reduced catalytic ability with a cyclic secondary amine.

<2> The method according to <1>, wherein the titanosilicate catalyst is a titanosilicate having pores not smaller than a 12-membered oxygen ring.

<3> The method according to <1> or <2>, wherein the titanosilicate catalyst is a Ti-MWW precursor.

<4> The method according to any one of <1> to <3>, wherein the titanosilicate catalyst is a titanosilicate catalyst obtained by mixing a silicon compound, boron compound, titanium compound and water with at least one structural directing agent selected from the group consisting of piperidine, hexamethyleneimine, N,N,N-trimethyl-1-adamantane ammonium salts and octyltrimethylammonium salts and heating a resulting mixture to obtain a lamellar compound, followed by removing the structural directing agent from the lamellar compound.

<5> The method according to any one of <1> to <4>, wherein the cyclic secondary amine is a compound represented by formula (I):

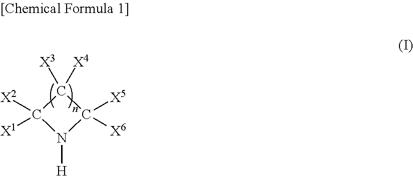

(wherein X¹, X², X³, X⁴, X⁵ and X⁶ each independently represent hydrogen, optionally substituted alkyl, optionally substituted alkenyl, optionally substituted alkynyl, optionally substituted cycloalkyl or optionally substituted aryl; and two groups from among X¹, X², X³, X⁴, X⁵ and X⁶ may be joined each other to form a hydrocarbon ring together with two adjacent carbon atoms of the ring; and n represents an integer of 0 to 9).

<6> The method according to any one of <1> to <5>, wherein the cyclic secondary amine is piperidine or hexamethyleneimine.

<7> The method according to any one of <1> to <6>, which further comprises holding a temperature of a mixture containing the titanosilicate having reduced catalytic ability and the cyclic secondary amine.

<8> The method according to any one of <1> to <7>, wherein mixing is carried out at a temperature in the range from 25 to 250° C.

<9> The method according to any one of <1> to <8>, wherein mixing is carried out in absence of hydrogen peroxide.

<10> The method according to any one of <1> to <9>, wherein the titanosilicate catalyst is used for obtaining an oxirane compound by reaction between hydrogen peroxide and a C2-C12 (herein, “Ci-Cii” means “having i to ii carbon atoms”) compound having a carbon-carbon double bond.

<11> The method according to <10>, wherein the oxirane compound is propylene oxide and the C2-C12 compound having a carbon-carbon double bond is propylene.

<12> The method according to any one of <1> to <11>, wherein the cyclic secondary amine is mixed in amount from 0.1 to 10 parts by weight per part by weight of the titanosilicate.

<13> A method for producing an oxirane compound, which comprises a step of reacting hydrogen peroxide with a C2-C12 compound having a carbon-carbon double bond, in the presence of a titanosilicate catalyst obtained by the method according to any one of <1> to <12>.

<14> A method for producing an oxirane compound, which comprises

a step of mixing a titanosilicate with a cyclic secondary amine in absence of hydrogen peroxide to obtain a mixture containing a regenerated titanosilicate,

a step of collecting the regenerated titanosilicate from the mixture, and

a step of reacting hydrogen peroxide with a C2-C12 compound having a carbon-carbon double bond, in the presence of the regenerated titanosilicate.

<15> The method according to <14>, wherein the oxirane compound is propylene oxide and the C2-C12 compound having a carbon-carbon double bond is propylene.

<16> A method for producing a regenerated titanosilicate catalyst, comprising a step of mixing a titanosilicate having reduced catalytic ability with a cyclic secondary amine.

<17> The method according to <16>, wherein the cyclic secondary amine is mixed in amount from 0.1 to 10 parts by weight per part by weight of the titanosilicate.

### Effects of Invention

According to the invention it is possible to provide a regeneration method that can regenerate a titanosilicate catalyst that has reduced catalytic ability.

### DESCRIPTION OF EMBODIMENTS

In the present specification, a titanosilicate having reduced catalytic ability (hereinafter also referred to as “degraded titanosilicate”) is one of the titanosilicates applicable for the invention, which shows a catalytic activity lower than a fresh titanosilicate.

The degraded titanosilicate is derived from a fresh titanosilicate or a regenerated titanosilicate, generally which has been used for the production of oxirane compounds or contacted with peroxides or glycol such as propylene glycol, or which has detracted from its original structure, for example, by continuously stirring it.

Here, the fresh titanosilicate means a titanosilicate which has not been applied to any reaction or mixed with the other compounds.

The regenerated titanosilicate means a titanosilicate obtained from the degraded titanosilicate, the catalytic activity of which is higher than that of the corresponding degraded titanosilicate.

The catalytic activity means a catalytic activity for the production of an oxirane compound described hereinafter. The catalytic activity can be determined by measuring yield of propylene oxide in the production of propylene oxide by reacting hydrogen peroxide with propylene. For determining the catalytic activity, the production of propylene oxide is preferably carried out under the conditions described hereinafter, for about 10 minutes to about 6 hours, preferably about 1 hour.

The degraded titanosilicate shows generally yield as high as about 2/10 to about 6/10 of the yield which the corresponding fresh titanosilicate shows, when the production of propylene oxide in the presence of the degraded titanosilicate is carried out under an identical condition to that in the presence of the corresponding fresh titanosilicate.

The regenerated titanosilicate generally shows yield as high as about 6/10 to about 12/10, preferably about 9/10 to about 11/10 of the yield which the corresponding fresh titanosilicate shows, when the production of propylene oxide in the presence of the regenerated titanosilicate is carried out under an identical condition to that in the presence of the corresponding fresh titanosilicate.

Herein, the “corresponding” refer to a case that a fresh titanosilicate is identical to a titanosilicate to be converted into a certain degraded or regenerated titanosilicate. Specifically, the “corresponding fresh titanosilicate” refers to a fresh titanosilicate which a degraded or regenerated titanosilicate is derived from. The “corresponding degraded titanosilicate” refers to a degraded titanosilicate which a regenerated titanosilicate is derived from.

The fresh, degraded and regenerated catalysts are collectively referred to as the titanosilicates applicable for the invention. The titanosilicate catalyst is a titanosilicate capable of catalyzing a reaction of hydrogen peroxide with a C2-C12 compound having a carbon-carbon double bond.

Here, “regenerating” or “regeneration” means increasing a catalytic activity for the reaction between hydrogen peroxide and a C2-C12 compound having a carbon-carbon double bond.

The titanosilicate applicable for the invention will now be described.

The “titanosilicate” includes any silicate with a tetracoordinated Ti (titanium atom), and it has a porous structure. According to the invention, “titanosilicate” refers to a titanosilicate essentially having a tetracoordinated Ti, and its ultraviolet and visible absorption spectrum in the wavelength range of 200 nm to 400 nm shows a maximum absorption peak in the wavelength range of 210 nm to 230 nm (see Chemical Communications 1026-1027(2002) FIG. 2(d), (e), for example). The ultraviolet and visible absorption spectrum can be measured by a diffuse reflection method using an ultraviolet and visible spectrophotometer equipped with a diffuse reflection sensor.

The titanosilicate applicable for the invention preferably has pores not smaller than a 10-membered oxygen ring, because this may reduce inhibition of contact between the reaction starting materials and the active sites inside the pores or may reduce restriction of movement of substances in the pore.

Throughout the present specification, the “pore” is an opening formed by Si—O bonds or Ti—O bonds. The pores may also be half-cup shaped pores, known as “side pockets”. In other words, it is not essential for the pores to pass through the primary particles of the titanosilicate.

The phrase “not smaller than a 10-membered oxygen ring” means that the number of oxygen atoms is 10 or greater in the ring structure (a) at a cross-section at the narrowest part of the pore or (b) at the entrance of the pore.

Generally, it can be confirmed that the titanosilicate has pores not smaller than a 10-membered oxygen ring, by analysis of the X-ray diffraction pattern, and if the structure is known it can be easily confirmed by comparison with the X-ray diffraction pattern.

The following titanosilicates 1 to 7 are examples of titanosilicates suitable for the invention.

1. Crystalline Titanosilicates with 10-Membered Oxygen Ring Pores:

TS-1 having an MFI structure (for example, U.S. Pat. No. 4,410,501), TS-2 having an MEL structure (for example, Journal of Catalysis 130, 440-446, (1991)), Ti-ZSM-48 having an MRE structure (for example, Zeolites 15, 164-170, (1995)) and Ti-FER having an FER structure (for example, Journal of Materials Chemistry 8, 1685-1686 (1998)), based on the IZA (International Zeolite Association) structure code.

2. Crystalline Titanosilicates with 12-Membered Oxygen Ring Pores:

Ti-Beta having a BEA structure (for example, Journal of Catalysis 199, 41-47, (2001)), Ti-ZSM-12 having an MTW structure (for example, Zeolites 15, 236-242, (1995)), Ti-MOR having an MOR structure (for example, The Journal of Physical Chemistry B 102, 9297-9303, (1998)), Ti-ITQ-7 having an ISV structure (for example, Chemical Communications 761-762, (2000)), Ti-MCM-68 having an MSE structure (for example, Chemical Communications 6224-6226, (2008)), and Ti-MWW having an MWW structure (for example, Chemistry Letters 774-775, (2000)).

3. Crystalline Titanosilicates with 14-Membered Oxygen Ring Pores:

Ti-UTD-1 having a DON structure (for example, Studies in Surface Science and Catalysis 15, 519-525, (1995)).

4. Lamellar Titanosilicates with 10-Membered Oxygen Ring Pores:

Ti-ITQ-6 (for example, Angewandte Chemie International Edition 39, 1499-1501, (2000)).

5. Lamellar Titanosilicates with 12-Membered Oxygen Ring Pores:

Ti-MWW precursors (for example, EP 1731515A1), Ti-YNU-1 (for example, Angewandte Chemie International Edition 43, 236-240(2004)), Ti-MCM-36 (for example, Catalysis Letters 113, 160-164(2007)), Ti-MCM-56 (for example, Microporous and Mesoporous Materials 113, 435-444(2008)).

6. Mesoporous Titanosilicates:

Ti-MCM-41 (for example, Microporous Materials 10, 259-271, (1997)), Ti-MCM-48 (for example, Chemical Communications 145-146, (1996)), Ti-SBA-15 (for example, Chemistry of Materials 14, 1657-1664, (2002)).

7. Silylated Titanosilicates:

Compounds that are silylated forms of the titanosilicates of 1 to 6 above, such as silylated Ti-MWW.

“Lamellar titanosilicates” means a titanosilicate with a lamellar structure, such as lamellar precursors of crystalline titanosilicates or titanosilicates that result from expansion between layers of crystalline titanosilicates. A lamellar structure can be confirmed with an electron microscope or by measurement of the X-ray diffraction pattern.

A lamellar precursor is a titanosilicate that is converted to a crystalline titanosilicate by dehydrating condensation treatment or the like. That the lamellar titanosilicate has pores not smaller than a 12-membered oxygen ring can be easily confirmed from the structure of the corresponding crystalline titanosilicate.

“Mesoporous titanosilicates” is a term including all titanosilicates having regular mesopores. Regular mesopores denotes a structure with a regularly repeating arrangement of mesopores.

A mesopore is a pore having a pore size of 2 nm to 10 nm.

The silylated titanosilicates are obtained by treatment of a titanosilicate of 1 to 6 above using a silylating agent. Examples of silylating agents include 1,1,1,3,3,3-hexamethyldisilazane and trimethylchlorosilane (EP1488853A1, for example).

Preferred examples of titanosilicates include crystalline titanosilicates or lamellar titanosilicates having pores not smaller than a 12-membered oxygen ring, with Ti-MWW and Ti-MWW precursors being more preferred examples.

A Ti-MWW precursor is a titanosilicate having a lamellar structure, which is converted to Ti-MWW by dehydrating condensation. Such dehydrating condensation is usually carried out by heating the Ti-MWW precursor at about 300° C. to 650° C.

When it is a fresh titanosilicate, the Ti-MWW precursor can usually be synthesized by a method in which a lamellar compound produced by direct hydrothermal synthesis from a boron compound, a titanium compound, a silicon compound and a structural directing agent (or, “as-synthesized sample”) is contacted with an aqueous strong acid under reflux conditions, and the structural directing agent is removed to adjust the ratio of atomic number between silicon and nitrogen atoms (Si/N ratio) to 21 or greater (see Japanese Unexamined Patent Application Publication No. 2005-262164, for example).

The “as-synthesized sample” is converted to zeolite with an MWW structure by calcination, which is not titanosilicate and which has no peak at 210 nm to 230 nm in its ultraviolet and visible absorption spectrum.

Examples of the titanium compound used in above production method include titanium alkoxide such as tetra-n-butyl orthotitanate; peroxotitanate such as tetra-n-butylammonium peroxotitanate; titanium halide such as titanium tetrachloride; titanium acetate, titanium nitrate, titanium sulfate, titanium phosphate, titanium perchlorate and titanium dioxide. Titanium alkoxide is preferred titanium compound. The amount of titanium compound is generally 0.001 to 10 parts by weight, preferably 0.01 to 2 parts by weight with respect to 1 part by weight of the lamellar compound.

Examples of the silicon compound include tetraalkyl orthosilicate such as tetraethyl orthosilicate; and silica.

Examples of the boron compound include boric acid.

The boron compound and the silicon compound may be used in almost the same amount.

Examples of the structural directing agent, i.e., structural directing agents capable of forming zeolite with an MWW structure, include piperidine, hexamethyleneimine, N,N,N-trimethyl-1-adamantane ammonium salts (for example, N,N,N-trimethyl-1-adamantane ammonium hydroxide, N,N,N-trimethyl-1-adamantane ammonium iodide and the like) and octyltrimethylammonium salts (for example, octyltrimethylammonium hydroxide, octyltrimethylammonium bromide and the like) (see Chemistry Letters 916-917 (2007), for example). Preferred structural directing agent among these are piperidine and hexamethyleneimine. Any one kind of these compounds may be used, or two or more kinds of that may be used in admixture in any desired proportion.

When it is a fresh titanosilicate, the Ti-MWW precursor can also be produced by a method including a step in which a lamellar borosilicate obtained by heating a mixture comprising a structural directing agent, boron compound, silicon compound and water, preferably after removing the structural directing agent by contact with an acid or the like, is calcined to obtain B-MWW, the boron is removed from the obtained B-MWW with an acid or the like, and then a structural directing agent, a titanium compound and water are added and the mixture is heated to obtain a lamellar compound, which is then contacted with 6M nitric acid to remove the structural directing agent (see Chemical Communication 1026-1027(2002), for example).

When it is a fresh titanosilicate, the Ti-MWW precursor can also be produced by a method including a step in which a mixture comprising a structural directing agent, a boron compound, a silicon compound and water is heated to obtain a lamellar borosilicate, which is then contacted with a titanium source and an inorganic acid to remove the structural directing agent. A Ti-MWW precursor obtained from a lamellar compound obtained by such a method as described above is calcined at a temperature of 530° C. for conversion to Ti-MWW having a peak at 210 nm to 230 nm in the ultraviolet and visible absorption spectrum.

The present invention is suitable for regeneration of Ti-MWW precursors with Si/N ratios of generally 5 to 20.

Ti-MWW precursors with Si/N ratios of 5 to 20 (hereinafter referred to simply as “precursors”) will now be described. The elements in a sample can be analyzed by the following common method. Ti (titanium), Si (silicon) and B (boron) can be measured by ICP luminescence analysis, and N (nitrogen) can be measured by oxygen circulation combustion/TCD detection (throughout the present specification, this is accomplished using a SUMIGRAPH NCH-22F (by Sumika Chemical Analysis Service, Ltd.)).

When it is a fresh titanosilicate, the Ti-MWW precursor can be obtained by contacting a titanosilicate having an X-ray diffraction pattern having the following values, with a structural directing agent capable of forming zeolite having an MWW structure.

X-ray diffraction pattern

(Lattice Spacing D/Angstrom)

12.4±0.8

10.8±0.3

9.0±0.3

6.0±0.3

3.9±0.1

3.4±0.1

These X-ray diffraction patterns can be measured using a common X-ray diffraction apparatus with copper K-alpha radiation.

When it is a fresh titanosilicate, the Ti-MWW precursor with Si/N ratios of generally 5 to 20 can be produced by a synthesis method comprising a step of contacting the titanosilicate as mentioned above Si/N ratio of which is 21 or greater with the structural directing agent. The amount of structural directing agent used is in the range of, generally 0.001 to 100 part by weight, preferably 0.1 to 10 part by weight relative to part by weight of the titanosilicate Si/N ratio of which is 21 or greater.

The step of contacting can be accomplished by a method of placing them in a sealed container such as an autoclave and applying pressure while heating, or a method of mixing them in a glass flask under air, with or without stirring. The temperature may be in the range of 0° C. to 250° C., preferably in the range of 25° C. to 250° C., more preferably in the range of 50° C. to 200° C. The pressure for contact may be about 0 to 10 MPa as the gauge pressure. Following contact, the obtained precursor will usually be separated by filtration. Depending on the need, water or the like may be used for rinsing to obtain a precursor with a Si/N ratio in the range of 5 to 20. The rinsing may be carried out with appropriate adjustment of the amount of washing solution, or while monitoring the pH of the rinsing filtrate, as necessary.

The Ti-MWW precursor produced in this manner can be used as a catalyst for oxidation reaction or the like. The Si/N ratio of the precursor is in the range of 5 to 20, preferably the Si/N ratio is in the range of 7 to 16, more preferably the Si/N ratio is in the range of 8 to 14. The precursor may also be silylated using a silylating agent such as 1,1,1,3,3,3-hexamethyldisilazane, for example.

The method for regenerating a titanosilicate catalyst, that is the method for producing a regenerated titanosilicate catalyst, comprises a step of mixing a degraded titanosilicate with a cyclic secondary amine. Hereinafter, such step is sometimes referred to as “mixing step”.

When the degraded titanosilicate is a titanosilicate which has been used for the production of oxirane compounds, it may be subjected to the mixing step in its form used for the reaction including a reaction filler such as alumina. When the degraded titanosilicate is a titanosilicate which has been used for the production of oxirane compounds, it is preferably subjected to the mixing step after isolating it.

The cyclic secondary amine is an optionally substituted compound having an alicyclic hydrocarbon ring in which at least one carbon atom of the ring has been replaced by an imino group (-NH-).

Examples for the cyclic secondary amine include compounds represented by formula (I):

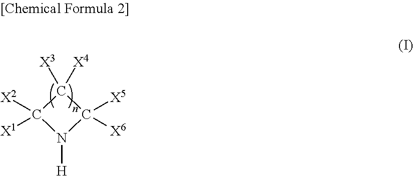

(wherein X¹, X², X³, X⁴, X⁵ and X⁶ each independently represent hydrogen, optionally substituted alkyl, optionally substituted alkenyl, optionally substituted alkynyl, optionally substituted cycloalkyl or optionally substituted aryl; and two groups from among X¹, X², X³, X⁴, X⁵ and X⁶ may be joined each other to form a hydrocarbon ring together with two adjacent carbon atoms of the ring; and n represents an integer of 0 to 9).

Examples of alkyl groups include C1-C12 straight-chain or branched alkyl groups such as methyl, ethyl, propyl, butyl, pentyl and hexyl.

Examples of alkenyl groups include C2-C12 alkenyl groups such as vinyl, propenyl, butenyl, pentenyl and hexenyl.

Examples of alkynyl groups include C2-C12 alkynyl groups such as ethynyl, propynyl, butynyl, pentynyl and hexynyl.

Examples of cycloalkyl groups include C3-C12 cycloalkyl groups such as cyclopropyl, cyclobutyl, cyclopentyl and cyclohexyl.

Examples of aryl groups include C6-C12 aryl groups such as phenyl and naphthyl.

The alkyl, alkenyl and alkynyl groups may be optionally substituted with one or more substituents selected from Group A defined below.

The cycloalkyl groups may be optionally substituted with one or more substituents selected from Group B defined below.

The aryl groups may be optionally substituted with one or more substituents selected from Group C defined below.

Group A: The group consisting of cycloalkyl, aryl, alkoxy, formyl, carboxy, alkoxycarbonyl, hydroxyl, mercapto, halogens and amino.

Group B: The group consisting of alkyl, alkenyl, alkynyl, cycloalkyl, aryl, alkoxy, formyl, carboxy, alkoxycarbonyl, hydroxyl, mercapto, halogens and amino.

Group C: The group consisting of alkyl, alkenyl, alkynyl, cycloalkyl, C1-C12 alkoxy, formyl, carboxy, alkoxycarbonyl, hydroxyl, mercapto, halogens and amino.

The alkyl, cycloalkyl, alkenyl, alkynyl and aryl groups in Groups A, B and C represent the same groups as mentioned above.

Examples of alkoxy groups include C1-C12 alkoxy groups such as methoxy, ethoxy, propoxy, butoxy, pentyloxy and hexyloxy.

Examples of alkoxycarbonyl groups include groups comprising the alkoxy and carbonyl groups mentioned above.

Specific examples of compounds represented by formula (I) include compounds wherein one methylene group of a C3-C12 alicyclic hydrocarbon is replaced by an imino group (—NH—), such as aziridine, azetidine, pyrrolidine, piperidine, hexamethyleneimine or azocane (compounds of formula (I) wherein n is 0 to 9 and X¹, X², X³, X⁴ and X⁵ and X⁶ are all hydrogen); compounds of formula (I) wherein two groups selected from among X¹, X², X³, X⁴, X⁵ and X⁶ are bonded to two adjacent carbon atoms and those groups are bonded together to form a hydrocarbon ring together with the two carbon atoms, such as 1,2,3,4-tetrahydroquinaldine or 1,2,3,4-tetrahydroquinoline; and compounds of formula (I) wherein X¹, X², X³, X⁴, X⁵ or X⁶ is alkyl or carboxy, such as 2-methylpiperidine, 4-methylpiperidine, 1,2,2,6,6-pentamethylpiperidine, 3,5-dimethylpiperidine, 2,6-dimethylpiperidine, 2-ethylpiperidine, 4-ethylpiperidine, 1,2,2,6,6-pentaethylpiperidine, 3,5-diethylpiperidine, 2,6-diethylpiperidine or 2,2,6,6-tetramethylpiperidine. Preferred examples of cyclic secondary amines include piperidine and hexamethyleneimine.

The cyclic secondary amine may form a salt with an acid. The acid includes hydrochloric acid, sulfuric acid, nitric acid, phosphoric acid, carbonic acid, a C1-C12 fatty acid, a C1-C12 sulfonic acid. Nitric acid is a preferred acid.

Examples of preferred cyclic secondary amine salts include acid salts (for example, hydrochlorides, sulfuric acid salts, nitric acid salts, phosphoric acid salts, carbonates, C1-C12 fatty acid salts, C1-C3 alkylsulfuric acid salts and sulfonic acid salts) of piperidine or hexamethyleneimine.

The lower limit for the amount of cyclic secondary amines is generally 0.01 part by weight, preferably 0.1 part by weight, more preferably 1 part by weight and even more preferably 2 parts by weight, per part by weight of the degraded titanosilicate.

The upper limit is 100 parts by weight, preferably 50 parts by weight, more preferably 10 parts by weight and even more preferably 5 parts by weight, per part by weight of the degraded titanosilicate.

The step is preferably carried out in the presence of a solvent.

The solvent includes water and an organic solvent. Examples of organic solvents include C1-C6 alcohols such as methanol and ethanol, C3-C6 ketone solvents such as acetone, methyl ethyl ketone and methyl isobutyl ketone, nitrile solvents such as acetonitrile, ether solvents such as diethyl ether, tetrahydrofuran and polypropyleneglycol dimethyl ether, aliphatic hydrocarbons such as pentane, hexane and heptane, aromatic hydrocarbons such as benzene, toluene and xylene, halogenated hydrocarbons such as chloroform, methylene chloride and chlorobenzene, and ester solvents such as ethyl acetate, butyl acetate and polypropyleneglycol diacetate, as well as mixtures of the foregoing.

The solvent is preferably water or a mixture of water and C1-C6 alcohols, more preferably water.

The step is generally carried out in absence of hydrogen peroxide.

The step is carried out preferably in absence of hydrogen peroxide, more preferably in absence of hydrogen peroxide and a compound having a carbon-carbon double bond.

The mixing step is carried out at a temperature in the range generally from 25 to 250° C., preferably from 50 to 200° C., more preferably from 100 to 200° C.

The pressure at the mixing step is preferably from ordinary pressure to pressurization of about 0 to 10 MPa, as the gauge pressure.

The method for regenerating a titanosilicate catalyst may further comprise a step of holding a temperature of a mixture of the titanosilicate having reduced catalytic ability and the cyclic secondary amine, preferably in the range of from 25 to 250° C., more preferably from 50 to 200° C., still more preferably from 100 to 200° C.

The holding time for the step of holding a temperature of the mixture is preferably 10 minutes, more preferably 1 hour, still more preferably 2 hours, still further more preferably 10 hours, and particularly more preferably 12 hours. The holding time is also preferably 120 hours or less, more preferably 72 hours or less, still more preferably 30 hours or less, and further more preferably 24 hour or less.

The regenerated titanosilicate is separated or collected by filtration, for example. If necessary, the separated regenerated titanosilicate may be subjected to post-treatment such as rinsing and drying.

Titanosilicate catalyst is generally used for obtaining an oxirane compound by reaction between hydrogen peroxide and a compound having a carbon-carbon double bond.

The present application encompasses a method for producing an oxirane compound which comprises a step of mixing a titanosilicate with a cyclic secondary amine in absence of hydrogen peroxide to obtain a mixture to containing a regenerated titanosilicate, a step of collecting the regenerated titanosilicate from the mixture, and a step of reacting hydrogen peroxide with a C2-C12 compound having a carbon-carbon double bond, in the presence of the regenerated titanosilicate.

The step of obtaining an oxirane compound by reaction between hydrogen peroxide and a compound having a carbon-carbon double bond in the presence of a titanosilicate catalyst to be used for the invention (hereinafter also referred to as “oxidation step”) will now be explained.

The compound having a carbon-carbon double bond includes preferably a C2-C12 alkene or C4-C12 cycloalkene compound, where the alkenes and cycloalkenes may be optionally substituted compounds. C2-C12 compounds having carbon-carbon double bonds are also collectively referred to as “olefins”.

The substituents on the olefins may be hydroxyl groups, halogen atoms, carbonyl, alkoxycarbonyl, cyano, nitro groups and the like.

Examples of C2-C10 alkenes include ethylene, propylene, butene, pentene, hexene, heptene, octene, nonene, decene, 2-butene, isobutene, 2-pentene, 3-pentene, 2-hexene, 3-hexene, 4-methyl-1-pentene, 2-heptene, 3-heptene, 2-octene, 3-octene, 2-nonene, 3-nonene, 2-decene and 3-decene.

Examples of C4-C10 cycloalkenes include cyclobutene, cyclopentene, cyclohexene, cycloheptene, cyclooctene, cyclononene and cyclodecene.

Propylene is a more preferred olefin.

The oxidation step may be reaction with an olefin after the step of contacting the titanosilicate catalyst and hydrogen peroxide (hereinafter also referred to as “contacting step”).

The hydrogen peroxide used in the contacting step is preferably a hydrogen peroxide solution. The concentration of the hydrogen peroxide solution may be in the range of 0.0001 wt % to 50 wt %, for example. The hydrogen peroxide solution may be, for example, a solution in water alone, a solution in one of the aforementioned organic solvents, or a mixed solution with water and an organic solvent.

The temperature for the contacting step is generally in the range of 0 to 100° C., and preferably in the range of 0 to 60° C.

Hydrogen peroxide is used for olefin oxidation reaction at a concentration in the range of 0.0001 wt % to 100 wt %, for example. The amount of hydrogen peroxide with respect to the olefin may be in the range of 1000:1 to 1:1000, for example, as the olefin:hydrogen peroxide molar ratio. The olefin:hydrogen peroxide molar ratio is generally in the range of 10:1 to 0.1:1, and is more preferably in the range of 5:1 to 0.1:1, and is more preferably in the range of 2:1 to 0.5:1.

The hydrogen peroxide used may be produced by a known method, and it may be hydrogen peroxide produced from oxygen and hydrogen in the reactor for the oxidation reaction.

Examples of precious metal catalysts to be used in the reaction for production of hydrogen peroxide from oxygen and hydrogen include precious metals such as palladium, platinum, ruthenium, rhodium, iridium, osmium and gold, as well as alloys and mixtures of these metals. Preferred precious metals are palladium, platinum and gold. Palladium is even more preferred as a precious metal. The palladium may be, for example, palladium colloid (see Japanese Unexamined Patent Application Publication No. 2002-294301, Example 1, for example). The precious metal catalyst used may be a precious metal compound that is converted to the precious metal by reduction in an oxidation reaction system, with the preferred precious metal compounds being palladium compounds. For example, when palladium is used as the precious metal catalyst it may be as a mixture of palladium with a metal other than palladium, such as platinum, gold, rhodium, iridium or osmium. Preferred metals other than palladium include gold and platinum.

Examples of palladium compounds include tetravalent palladium compounds such as sodium hexachloropalladate(IV) tetrahydrate and potassium hexachloropalladate(IV), and divalent palladium compounds such as palladium(II) chloride, palladium(II) bromide, palladium(II) acetate, palladium(II) acetylacetonate, dichlorobis(benzonitrile)palladium(II), dichlorobis(acetonitrile)palladium(II), dichloro(bis(diphenylphosphino)ethane)palladium(II), dichlorobis(triphenylphosphine)palladium(II), dichlorotetraminepalladium(II), dibromotetraminepalladium(II), dichloro(cycloocta-1,5-diene)palladium(II) and palladium(II) trifluoroacetate.

The precious metal is preferably used by being loaded on a support. The precious metal may be used by loading on the titanosilicate catalyst, or it may be used by loading on an oxide such as silica, alumina, titania, zirconia or niobia; a hydroxide of niobic acid, zirconic acid, tungstic acid, titanic acid or the like; carbon; or a mixture of the foregoing. When a precious metal other than the titanosilicate catalyst is supported, the precious metal-loaded support may be mixed with the titanosilicate catalyst and the mixture used as the catalyst. Carbon is a preferred support among supports other than titanosilicate catalysts. Active carbon, carbon black, graphite and carbon nanotubes are known forms of carbon supports.

The method of preparing the precious metal catalyst may be a known method in which, for example, the precious metal compound is loaded on the support and then reduced. Loading of the precious metal compound can be accomplished by a known method such as impregnation.

When a reducing gas is used for the reduction, the loaded precious metal compound in solid form may be packed into an appropriate packed tube and the reducing gas injected into the packed tube, for reduction by a simple procedure. The reducing gas may be, for example, hydrogen, carbon monoxide, methane, ethane, propane, butane, ethylene, propylene, butene, butadiene, or a mixed gas comprising two or more of the foregoing. Hydrogen is preferred among these. The reducing gas may also be diluted with a diluent gas that is nitrogen, helium, argon or water vapor (steam), or a mixture of two or more of the foregoing.

The precious metal catalyst comprises the precious metal in a range of 0.01 to 20 wt % and preferably 0.1 to 5 wt %. The amount (lower limit) for use of the precious metal is, for example, at least 0.00001 parts by weight, preferably at least 0.0001 part by weight and more preferably at least 0.001 part by weight, with respect to 1 part by weight of the titanosilicate catalyst. The amount (upper limit) for use of the precious metal is, for example, no greater than 100 parts by weight, preferably no greater than 20 parts by weight and more preferably no greater than 5 parts by weight, with respect to 1 part by weight of the titanosilicate.

The oxidation step may be accomplished by, for example, by a method of mixing an olefin with the solution containing hydrogen peroxide obtained in the contacting step, or a method of oxidizing the olefin while generating hydrogen peroxide in a mixture of oxygen, hydrogen and the olefin in the presence of the titanosilicate catalyst.

The oxidation step is preferably carried out in a liquid phase containing a solvent. The solvent may be water, an organic solvent or a mixture of the two.

Examples of organic solvents include alcohol solvents, ketone solvents, nitrile solvents, ether solvents, aliphatic hydrocarbons, aromatic hydrocarbons, halogenated hydrocarbons, ester solvents, and mixtures of the foregoing.

Examples of alcohol solvents include C1-C8 aliphatic alcohols such as methanol, ethanol, isopropanol and t-butanol; and C2-C8 glycols such as ethylene glycol and propylene glycol. Examples of preferred alcohol solvents include C1-C4 monohydric alcohols, with t-butanol being more preferred.

Examples of aliphatic hydrocarbons include C5-C10 aliphatic hydrocarbons such as hexane and heptane. Examples of aromatic hydrocarbons include C6-C15 aromatic hydrocarbons such as benzene, toluene and xylene.

Examples of nitrile solvents include C2-C4 alkylnitriles such as acetonitrile, propionitrile, isobutyronitrile and butyronitrile, as well as benzonitrile, with acetonitrile being preferred.

The solvent used for the oxidation step is preferably a C1-C4 monohydric alcohol or acetonitrile, from the viewpoint of catalytic activity and selectivity.

The lower limit for the reaction temperature in the oxidation step is generally 0° C., preferably 40° C. The upper limit for the reaction temperature in the oxidation step may be 200° C., for example, and is preferably 150° C., and more preferably 80° C.

The lower limit for the reaction pressure (gauge pressure) in the oxidation step is 0.1 MPa, for example, and it is preferably 1 MPa, more preferably 20 MPa and even more preferably 10 MPa.

A known method such as distilling separation may be used to obtain an oxirane compound from the product of the oxidation step.

The amount of olefin used in the oxidation step generally differs depending on the type of olefins and on the reaction conditions, but it is preferably 0.01 part by weight or greater and more preferably 0.1 part by weight or greater, with respect to 100 parts by weight as the total of the solvent in the liquid phase. The amount of organic compound is preferably 1000 or less parts by weight and more preferably 100 or less parts by weight with respect to 100 parts by weight as the total of the solvent in the liquid phase.

The amount of titanosilicate catalyst in the oxidation step may be appropriately selected according to the type of reaction, and its lower limit may be 0.01 part by weight, preferably 0.1 part by weight and more preferably 0.5 part by weight while its upper limit may be 20 parts by weight, preferably 10 parts by weight and more preferably 8 parts by weight, with respect to 100 parts by weight as the total of the solvent used in the oxidation step.

A buffering agent is preferably present for the oxidation step, because it can prevent reduction in catalytic activity, further increase the catalytic activity, and will tend to improve the oxygen and hydrogen utilization efficiency.

The buffering agent is preferably added during the oxidation step by dissolution in the solution, but when the hydrogen peroxide produced in the same step is used as the oxidizing agent, it may be included in a portion of the precious metal catalyst used for production of the hydrogen peroxide. For example, the method may involve loading an amine complex such as Pd-tetramine chloride on a support by impregnation and then reducing it, leaving residual ammonium ion, and generating a buffering agent during oxidation reaction. The amount of buffering agent added may be in the range of 0.001 mmol to 100 mmol, for example, to 1 kg of solvent.

The buffering agent may be one comprising 1) an anion selected from the group consisting of sulfate ion, hydrogensulfate ion, carbonate ion, hydrogencarbonate ion, phosphate ion, hydrogenphosphate ion, dihydrogenphosphate ion, hydrogenpyrophosphate ion, pyrophosphate ion, halide ions, nitrate ion, hydroxide ion and C1-C10 carboxylate ions, and 2) a cation selected from the group consisting of ammonium, C1-C20 alkylammonium, C7-C20 alkylarylammonium, alkali metal and alkaline earth metal ions.

Examples of C1-C10 carboxylate ions include acetate ion, formate ion, acetate ion, propionate ion, butyrate ion, valerate ion, caproate ion, caprylate ion, caprate ion and benzoate ion.

Examples of alkylammonium ions include tetramethylammonium, tetraethylammonium, tetra-n-propylammonium, tetra-n-butylammonium and cetyltrimethylammonium, and examples of alkali metal and alkaline earth metal cations include lithium cation, sodium cation, potassium cation, rubidium cation, cesium cation, magnesium cation, calcium cation, strontium cation and barium cation.

Preferred buffering agents include ammonium salts of inorganic acids such as ammonium sulfate, ammonium hydrogensulfate, ammonium carbonate, ammonium hydrogencarbonate, diammonium hydrogenphosphate, ammonium dihydrogenphosphate, ammonium phosphate, ammonium hydrogenpyrophosphate, ammonium pyrophosphate, ammonium chloride and ammonium nitrate, and ammonium salts of C1-C10 carboxylic acids such as ammonium acetate, with ammonium dihydrogenphosphate being a preferred example of an ammonium salt.

When hydrogen peroxide is synthesized from oxygen and hydrogen in the same reaction system as the oxidation reaction, for use in the oxidation step, it is preferred to add a quinoid compound to the oxidation step since this will tend to further increase selectivity for the oxirane compound.

Examples of quinoid compounds include compounds represented by formula (1):

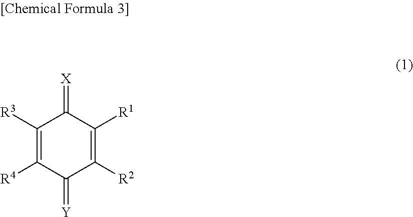

(wherein R¹, R², R³ and R⁴ each independently represent hydrogen, or R¹ and R² or R³ and R⁴ bond together to form an optionally substituted benzene ring or optionally substituted naphthalene ring together with the carbon atoms to which R¹, R², R³ and R⁴ are bonded, and X and Y each independently represent an oxygen atom or NH group).

Examples of compounds of formula (1) include:

1) quinone compounds (1A) of formula (1) wherein R¹, R², R³ and R⁴ are hydrogen and X and Y are both oxygen atoms,

2) quinoneimine compounds (1B) of formula (1) wherein R¹, R², R³ and R⁴ are hydrogen, X is an oxygen atom and Y is an NH group,

3) quinonediimine compounds (1C) of formula (1) wherein R¹, R², R³ and R⁴ are hydrogen and X and Y are NH groups, and

4) compounds represented by formula (2), which are compounds of formula (1) wherein R¹ and R² are bonded together and R³ and R⁴ are bonded together, to form an optionally substituted benzene ring together with the carbon atoms to which R², R³ and R⁴ are bonded.

Examples of other compounds represented by formula (1) include anthraquinone compounds represented by formula (2):

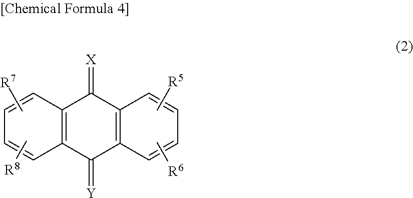

(wherein X and Y have the same meanings as above, and R⁵, R⁶, R⁷ and R⁸ each independently represent hydrogen, hydroxyl or alkyl (for example, a C1-C5 alkyl group such as methyl, ethyl, propyl, butyl or pentyl)).

X and Y in the compounds represented by formula (1) are preferably oxygen atoms.

Examples of quinoid compounds include quinone compounds such as benzoquinone, naphthoquinone and 9,10-phenanthraquinone, and anthraquinones, including 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone and 2-s-amylanthraquinone; polyalkylanthraquinone compounds such as 1,3-diethylanthraquinone, 2,3-dimethylanthraquinone, 1,4-dimethylanthraquinone and 2,7-dimethylanthraquinone, and polyhydroxyanthraquinone compounds such as 2,6-dihydroxyanthraquinone.

Preferred compounds represented by formula (1) include anthraquinone and 2-alkylanthraquinone compounds (compounds of formula (2) wherein X and Y are oxygen atoms, R⁵ is an alkyl group, R⁶ is hydrogen and R⁷ and R⁸ are hydrogen).

The amount of quinoid compound used in the oxidation step may be in the range of 0.001 mmol to 500 mmol, for example, and is preferably in the range of 0.01 mmol to 50 mmol, to 1 kg of the solvent in the liquid phase.

A salt comprising ammonium, alkylammonium or alkylarylammonium may also be added during the oxidation step.

A quinoid compound can be prepared by oxidation of the dihydro form of the quinoid compound with oxygen in the oxidation step. For example, hydroquinone or a hydrogenated quinoid compound such as 9,10-anthracenediol may be added to the liquid phase and oxidized by oxygen in the reactor to generate a quinoid compound.

Examples of dihydro forms of quinoid compounds include compounds represented by formula (3):

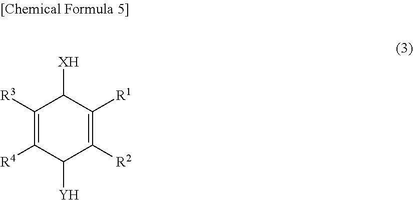

(wherein R¹, R², R³, R⁴, X and Y have the same meanings as above),

which are dihydro forms of compounds represented by formula (1), and compounds represented by formula (4):

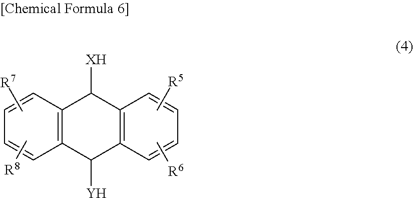

(wherein X, Y, R⁵, R⁶, R⁷ and R⁸ have the same meanings as above),

which are dihydro forms of compounds represented by formula (2).

X and Y in formula (3) and formula (4) are preferably oxygen atoms.

Preferred dihydro forms of quinoid compounds are dihydro forms of the preferred quinoid compounds mentioned above.

There are no particular restrictions on the reactor used for the oxidation step employing a prepared hydrogen peroxide, but it is preferred to use a circulating fixed bed reactor, circulating slurry complete mixing apparatus or the like, in an oxidation step that includes a contacting step.

For an oxidation step that includes a contacting step, the partial pressure ratio of oxygen and hydrogen supplied to the reactor may be, for example, in the range of oxygen:hydrogen=1:50 to 50:1, and it is preferably in the range of oxygen:hydrogen=1:2 to 10:1. If the oxygen partial pressure is at least oxygen:hydrogen=1:50 the oxirane compound production rate will tend to be increased, and this is therefore preferred, while if the oxygen partial pressure is no higher than oxygen:hydrogen=50:1 there will tend to be lower generation of by-products in which the carbon-carbon double bonds of the olefin are reduced by hydrogen, resulting in increased oxirane compound selectivity, and this is also preferred.

The oxygen and hydrogen gas used in the oxidation step may be diluted. Examples of gases to be used for dilution include nitrogen, argon, carbon dioxide, methane, ethane and propane.

The oxygen starting material may be oxygen gas or air, for example. The oxygen gas used may be oxygen gas produced by an inexpensive pressure swing process, or if necessary it may be high purity oxygen gas produced by cryogenic separation.

The oxirane compound includes an epoxide compound having 2 to 12 carbon atoms. Preferable examples of the epoxide compound include ethylene oxide, propylene oxide, butene oxide, pentene oxide, hexene oxide, heptene oxide, octene oxide, nonene oxide, decene oxide.

Propylene oxide is a more preferred oxirane compound.

The titanium silicate catalyst that has been regenerated by the regeneration method of the invention may be used for reaction between hydrogen peroxide and a compound having a carbon-carbon double bond to obtain an oxirane compound, in the same manner as described above.

### EXAMPLES

The present invention will now be explained by examples.

(Analyzer Used for Examples)

### [Elemental Analysis]

The Ti (titanium), Si (silicon) and B (boron) contents were determined by alkali fusion-nitric acid dissolution-ICP emission analysis. The N (nitrogen) content was measured with an oxygen circulating combustion/TCD detection system employing a SUMIGRAPH (product of Sumitomo Chemical Analysis Center).

[Powder X-Ray Diffraction (XRD)]

The powder X-ray diffraction pattern of the sample was measured with the following apparatus and conditions.

Apparatus: RINT2500 V by Rigaku Corp.

Line source: Cu Kα-rays

Output: 40 kV-300 mA

Scanning zone: 2θ=0.75 to 30°

Scanning rate: 1°/min

[Ultraviolet and Visible Absorption Spectrum (UV-Vis)]

The sample was thoroughly pulverized with an agate mortar and then pelletized (7 mmφ) to prepare a measuring sample, and the ultraviolet and visible absorption spectrum of the measuring sample was measured with the following apparatus and conditions.

Apparatus: Diffuse reflection sensor (Praying Mantis, by HARRICK).

Accessory: Ultraviolet and visible spectrophotometer (V-7100 by JASCO Corp.)

Pressure: Atmospheric pressure

Measured value: Reflectance

Data acquisition time: 0.1 second

Band width: 2 nm

Measuring wavelength: 200 to 900 nm

Slit height: Semi-open

Data acquisition interval: 1 nm

Baseline compensation (reference): BaSO₄ pellets (7 mmφ)

### [A Method for Checking the Ti-MWW Precursor]

When the X-ray diffraction pattern was similar to that in FIG. 1 in EP1731515A1, the sample was determined to be a Ti-MWW precursor.

### Reference Example 1

### [Preparation of Non-Degraded Ti-MWW Precursor (Applicable Titanosilicate Catalyst for the Invention)]

The titanosilicate used for the examples and comparative examples was prepared in the following manner. Specifically, 899 g of piperidine (Wako Pure Chemical Industries, Ltd.), 2402 g of ion-exchanged water, 46 g of tetra-n-butyl orthotitanate [TBOT] (Wako Pure Chemical Industries, Ltd.), 565 g of boric acid (Wako Pure Chemical Industries, Ltd.) and 410 g of fumed silica (product name: cab-o-sil M7D, Cabot Japan, KK.) were dissolved in an autoclave at 25° C. under an air atmosphere, and the solution was aged for 1.5 hours. The autoclave was then sealed and then heated to 150° C. over a period of 8 hours while stirring the obtained gel, after which it was held at the same temperature for 120 hours for hydrothermal synthesis to obtain a suspended solution.

The obtained suspended solution was filtered, and the recovered solid was rinsed with water until the washing solution reached nearly pH 10. The rinsed solid was then dried until almost no weight reduction was observed at 50° C., to obtain lamellar compound 1.

After adding 3750 mL of 2M nitric acid and 9.5 g of TBOT to 75 g of the lamellar compound 1, the mixture was warmed for 20 hours at the solvent reflux temperature. The reaction mixture was then filtered and the resulting solid was rinsed with water until the rinse solution reached near neutral, and then vacuum dried at 150° C. until virtually no further weight reduction was observed, to obtain 61 g of a white powder (solid product 1). The X-ray diffraction pattern of solid product 1 was measured, confirming that it had an MWW precursor structure. The same procedure was repeated twice to obtain a total of 122 g of Ti-MWW precursor 1.

The 61 g of Ti-MWW precursor 1 was calcined at 530° C. for 6 hours to obtain 55 g of solid product 2. The same procedure was repeated twice to obtain a total of 110 g of solid product 2. Solid product 2 has lattice spacing d=12.1, 10.8, 8.9, 6.1, 3.9, 3.4 angstrom in the X-ray diffraction pattern, and the results of ultraviolet and visible absorption spectrometry confirmed that it was Ti-MWW.

After suspending 300 g of piperidine, 600 g of ion-exchanged water and 80 g of solid product 2 in an autoclave at 25° C. under an air atmosphere, the suspension was aged for 1.5 hours. The autoclave was then sealed and heated to 170° C. over a period of 4 hours while stirring the obtained suspension, followed by holding it at the same temperature for 24 hours to obtain a suspended solution. The obtained suspended solution was filtered, and the recovered solid was rinsed with water until the rinsing solution reached nearly pH 9. The solid was dried until virtually no weight reduction was observed in a vacuum at 150° C., to obtain 79 g of a white powder (catalyst A). Based on the results of measuring the X-ray diffraction pattern and ultraviolet and visible absorption spectrum, catalyst A was determined to be a Ti-MWW precursor, with a titanium content of 2.08 wt % and a Si/N ratio of 9.2.

### Reference Example 2

[Preparation of Degraded Ti-MWW Precursor (Titanosilicate having Reduced Catalytic Ability)]

A degraded Ti-MWW precursor was prepared in the following manner. In a glass volumetric flask at 25° C. under an air atmosphere, suspended were 10 g of catalyst A prepared as described above, 75 g of propylene oxide (Wako Pure Chemical Industries, Ltd.), 150 g of propylene glycol (Wako Pure Chemical Industries, Ltd.) and 525 g of ion-exchanged water. The obtained suspension was then heated to 90° C. while stirring, and held at the same temperature for 24 hours to obtain a suspended solution. After filtering the suspended solution, the obtained solid was rinsed using 6 L of ion-exchanged water at 20° C. The solid was dried until virtually no weight reduction was observed in a vacuum at 150° C., to obtain 9 g of a white powder (catalyst B). Based on the results of measuring the X-ray diffraction pattern and ultraviolet and visible absorption spectrum, catalyst B was determined to be a Ti-MWW precursor, with a titanium content of 2.12 wt % and a Si/N ratio of 15.6.

### Example 1

### [Method for Regeneration of Degraded Ti-MWW Precursor]

The degraded Ti-MWW precursor prepared in Reference Example 2 was regenerated in the following manner. In a glass volumetric flask at 25° C. under an air atmosphere there were suspended 1 g of catalyst B, 3 g of piperidine (Wako Pure Chemical Industries, Ltd.) and 45 g of ion-exchanged water, and the obtained suspension was heated to 170° C. while stirring and held at the same temperature for 24 hours, to obtain a suspended solution. The obtained suspended solution was filtered, and the recovered solid was rinsed with water until the rinsing solution reached nearly pH 9. The solid was dried until virtually no weight reduction was observed in a vacuum at 150° C., to obtain 0.6 g of a white powder (catalyst C). Based on the results of measuring the X-ray diffraction pattern and ultraviolet and visible absorption spectrum, catalyst C was determined to be a Ti-MWW precursor, with a titanium content of 2.13 wt % and a Si/N ratio of 13.3.

### Example 2

### [Propylene Oxidation Using Regenerated Ti-MWW Precursor]

A 30 wt % hydrogen peroxide aqueous solution (Wako Pure Chemical Industries, Ltd.), acetonitrile (Nacalai Tesque, Inc.) and ion-exchanged water were used to prepare a 0.5 wt % hydrogen peroxide solution in an acetonitrile/water mixed solvent (weight ratio: 4/1). To a 100 mL stainless steel autoclave, 60 g of the prepared solution and 10 mg of catalyst C were added to give a mixture. The autoclave was then transferred to an ice bath, and 1.2 g of propylene was packed therein. The pressure in the autoclave was increased to 2 MPa (gauge pressure) with argon. The temperature of the autoclave was raised to 60° C. over a period of 15 minutes while stirring the mixture, and kept at that temperature for 1 hour for reaction. Following the reaction, the stirring was stopped and the autoclave was cooled on ice. After the ice-cooling, the liquid phase was analyzed by gas chromatography. According to the results, the propylene oxide yield was 86% based on the initial hydrogen peroxide amount.

### Reference Example 3

### [Propylene Oxidation Using Non-Degraded Ti-MWW Precursor]

Propylene oxide was produced by the same procedure as Example 2, except that catalyst A was used instead of catalyst C. According to the results, the propylene oxide yield was 85% based on the initial hydrogen peroxide amount.

### Reference Example 4

### [Propylene Oxidation Using Degraded Ti-MWW Precursor (Titanosilicate Catalyst)]

Propylene oxide was produced by the same procedure as Example 2, except that catalyst B was used instead of catalyst C. According to the results, the propylene oxide yield was 36% based on the initial hydrogen peroxide amount.

The results are summarized in Table 1. As seen from Table 1, the catalytic ability according to the invention was equivalent to that immediately after preparation of the catalyst.

TABLE 1

| Example | Titanosilicate catalyst | Yield* |
|---|---|---|
| Reference Example 3 | Non-degraded titanosilcate | 85% |
| | catalyst (catalyst A) | |
| Reference Example 4 | Degraded titanosilcate | 36% |
| | catalyst (catalyst B) | |
| Example 2 | Regenerated titanosilcate | 86% |
| | catalyst (catalyst C) | |

*1: Propylene oxide yield based on initial hydrogen peroxide amount.

### INDUSTRIAL APPLICABILITY

According to the invention it is possible to provide a regeneration method that can regenerate titanosilicate catalysts that have reduced catalytic ability.

1. A method for regenerating a titanosilicate catalyst, comprising a step of mixing a titanosilicate having reduced catalytic ability with a cyclic secondary amine.

2. The method according to claim 1, wherein the titanosilicate catalyst is a titanosilicate having pores not smaller than a 12-membered oxygen ring.

3. The method according to claim 1, wherein the titanosilicate catalyst is a Ti-MWW precursor.

4. The method according to claim 1, wherein the titanosilicate catalyst is a titanosilicate catalyst obtained by mixing a silicon compound, boron compound, titanium compound and water with at least one structural directing agent selected from the group consisting of piperidine, hexamethyleneimine, N,N,N-trimethyl-1-adamantane ammonium salts and octyltrimethylammonium salts and heating a resulting mixture to obtain a lamellar compound, followed by removing the structural directing agent from the lamellar compound.

5. The method according to claim 1, wherein the cyclic secondary amine is a compound represented by formula (I): 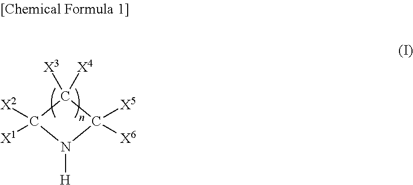
(wherein X¹, X², X³, X⁴, X⁵ and X⁶ each independently represent hydrogen, optionally substituted alkyl, optionally substituted alkenyl, optionally substituted alkynyl, optionally substituted cycloalkyl or optionally substituted aryl; and two groups from among X¹, X², X³, X⁴, X⁵ and X⁶ may be joined each other to form a hydrocarbon ring together with two adjacent carbon atoms of the ring; and n represents an integer of 0 to 9).

6. The method according to claim 1, wherein the cyclic secondary amine is piperidine or hexamethyleneimine.

7. The method according to claim 1, which further comprises holding a temperature of a mixture containing the titanosilicate having reduced catalytic ability and the cyclic secondary amine.

8. The method according to claim 1, wherein mixing is carried out at a temperature in the range from 25 to 250° C.

9. The method according to claim 1, wherein mixing is carried out in absence of hydrogen peroxide.

10. The method according to claim 1, wherein the titanosilicate catalyst is used for obtaining an oxirane compound by reaction between hydrogen peroxide and a C2-C12 compound having a carbon-carbon double bond.

11. The method according to claim 10, wherein the oxirane compound is propylene oxide and the C2-C12 compound having a carbon-carbon double bond is propylene.

12. The method according to claim 1, wherein the cyclic secondary amine is mixed in amount from 0.1 to 10 parts by weight per part by weight of the titanosilicate.

13. A method for producing an oxirane compound, which comprises a step of reacting hydrogen peroxide with a C2-C12 compound having a carbon-carbon double bond, in the presence of a titanosilicate catalyst obtained by the method according to claim 1.

14. A method for producing an oxirane compound, which comprises
a step of mixing a titanosilicate with a cyclic secondary amine in absence of hydrogen peroxide to obtain a mixture containing a regenerated titanosilicate,
a step of collecting the regenerated titanosilicate from the mixture, and
a step of reacting hydrogen peroxide with a C2-C12 compound having a carbon-carbon double bond, in the presence of the regenerated titanosilicate.

15. The method according to claim 14, wherein the oxirane compound is propylene oxide and the C2-C12 compound having a carbon-carbon double bond is propylene.

16. A method for producing a regenerated titanosilicate catalyst, comprising a step of mixing a titanosilicate having reduced catalytic ability with a cyclic secondary amine.

17. The method according to claim 16, wherein the cyclic secondary amine is mixed in amount from 0.1 to 10 parts by weight per part by weight of the titanosilicate.
